(19) United States
(12) Patent Application Publication
(10) Pub. No.: US 2014/0342928 A1
(43) Pub. Date: Nov. 20, 2014

(54) MEASURING METHOD FOR NUCLEIC ACID SAMPLES

(71) Applicant: CrackerBio, Inc., Hsinchu County (TW)

(72) Inventor: Chung-Fan Chiou, Hsinchu County (TW)

(73) Assignee: CrackerBio, Inc., Hsinchu County (TW)

(21) Appl. No.: 14/281,898

(22) Filed: May 20, 2014

Related U.S. Application Data
(60) Provisional application No. 61/855,573, filed on May 20, 2013.

(51) Int. Cl. C12Q 1/68; G01N 21/64; G01N 33/487

(52) U.S. Cl. CPC C12Q 1/6851; G01N 33/48792; G01N 21/64
USPC 506/9; 702/21

(57) ABSTRACT

A measuring method for nucleic acid samples is provided. The measuring method provides qualitative and quantitative analyses of different types of templates of divergent concentration ranges in one experiment. Hence, the same test panel of various assays may be performed in a single test slide.

### CROSS-REFERENCE TO RELATED APPLICATION

This application claims the priority benefits of U.S. provisional application Ser. No. 61/855,573, filed on May 20, 2013. The entirety of the above-mentioned patent applications is hereby incorporated by reference herein and made a part of this specification.

### BACKGROUND

1. Technical Field

The disclosure relates to a measuring method. Particularly, the disclosure relates to a measuring method for nucleic acid samples of divergent concentration ranges.

2. Related Art

In the field of molecular biology, quantitative or qualitative study of a variety of different nucleic acid targets, such as fragment of DNA or RNA or Micro RNA, may be required to investigate the specific sample. For example, it is common to test a sample for the expression levels of a group of genes. Also, several DNA assays may compose of a test panel for diagnosis purpose. A particular nucleic acid target presented in a biological specimen may have a typical concentration range. It is common that a group of nucleic acid species intended for a diagnostic panel has a broad range of concentrations.

Polymerase chain reaction (PCR) is one of the common methods for detecting the presence and also measuring the concentrations of the nucleic acid targets in samples. PCR reaction is usually considered as a wide dynamic range test method in comparing with most chemical analysis methods. The PCR dynamic range may span 5 orders of magnitude. However, still, in many situations, the nucleic acid targets of concern may have an even wider concentration range and are unable to “fit in” in a single test. At present, the only way to handle such a concentration range discrepancy is to dilute or concentrate the extracted nucleic acid solution in different dilutions or concentration ratios to fit in the dynamic range of PCR method. Performing dilution or concentration is neither time nor labour efficient.

### SUMMARY

The present invention utilizes an array type slide and real time fluorescence intensity detection technology, in combination with PCR method, to provide qualitative and quantitative test results.

The present invention provides a method of measuring concentrations of more than one nucleic acid targets (templates) in a sample. A test slide having a plurality of reaction wells is provided, and a sample needed to be detected or measured for a plurality of nucleic acid targets is provided. The plurality of reaction wells is divided into groups, and each group is assigned to detect one specific target. The primers and probes for detecting the specific target are dispensed into the respective group of wells before use. When the slide is used, the sample is loaded into each of the plurality of reaction wells. After submitting the test slide to predetermined thermal cycle conditions, the fluorescence intensity of each well at each cycle is measured and recorded. The obtained intensity data is then analysed to determine the concentration of each target.

Instead of (1) allocating equal number of wells to each target in the panel and (2) depositing equal sample volume to each group of wells, the present invention provides a method to determine (1) the optimized number of reaction wells to be assigned or allocated to each target and (2) the optimized sample volume to be deposited to each group of wells, based on the estimated concentration range of each target in a particular type of biological tissue or based on the intended concentration measurement range of the slide. The purpose is to minimize or even eliminate the steps of dilution/concentration when multiple targets with very different concentration ranges are encountered.

The present invention also provides a method to determine the concentration of a target, based on the changes in PCR fluorescence intensities among the reaction wells which are assigned to the target. The number of reaction wells used in this method usually is more than that needed for technical redundant, which is typically 2 to 10 redundants. The number of reaction wells used for measuring the target may be 50, 100, 500, or even several thousands. The method includes at least 3 types of concentration determination algorithm, which are Ct quantification, digital PCR quantification, a combination of both, and one pre-defined rule for choosing the appropriate concentration determination algorithm for a specific test. The pre-defined rule is based on the ratio or other statistical calculations of the number of reaction wells showing positive results to that of reaction wells showing negative results. The positive results or the negative result of a reaction well may be determined by whether the fluorescence intensity exceeds the select threshold or not.

According to embodiments of the present invention, each target has a possible concentration range with an upper boundary and a lower boundary. The lower boundary is used to calculate the minimum well number to be allocated to that target. The upper boundary is used to determine the maximum volume of the reaction well should be used.

More wells are assigned to those targets typically of low concentrations in the sample. For example, in gene expression profiling test, more wells are assigned to rare targets; and vice versa, less wells are assigned to those targets usually of high concentrations in the sample. Accordingly, the wells in the slide are used efficiently. More important, the dilution or concentration step is minimized or even eliminated.

According to embodiments of the present invention, the primers are filled along with fluorescent reporters to the wells before test. The filling step of these reagents may be done in the factory. The layout, i.e. the map of which well is assigned to which target, is associated with the slide. During data analysing process, the well fluorescence intensity data is grouped for analysis following the well assignment layout information.

In order to make the aforementioned and other features and advantages of the disclosure comprehensible, several exemplary embodiments accompanied with figures are described in detail below.

### BRIEF DESCRIPTION OF THE DRAWINGS

The accompanying drawings are included to provide a further understanding of the disclosure, and are incorporated in and constitute a part of this specification. The drawings illustrate embodiments of the disclosure and, together with the description, serve to explain the principles of the disclosure.

FIG. 1 shows a sample with three targets in which the concentration ranges respectively thereof are known.

### DETAILED DESCRIPTION OF DISCLOSED EMBODIMENTS

Traditional quantitative polymerase chain reaction (qPCR) method provides quantitative analysis of nucleic acid template(s) by recording the fluorescence intensity inside of a reaction well for each thermal cycle. In the qPCR method, the changes in the plotted statistical curves of the recorded fluorescence intensity are used to determine the copy number of the template(s) within the reaction well before the commencement of each thermal cycle. The initial copy number of the template(s) in the reaction well is used to determine the concentration of the template(s) within the tested sample. When using the qPCR method for quantitative analysis, it is required to have at least one copy of the template inside the reaction well for further replication, which leads to increased fluorescent intensity following more thermal cycles. In real-time qPCR, the Ct value is the number of the cycle at which the fluorescent intensity exceeds a threshold intensity or the cycle at which the fluorescent intensity starts to increase dramatically. The PCR cycle at which the sample reaches this level is called the cycle threshold (Ct). By comparing the Ct values of samples of unknown concentration with a series of standards or the pre-determined cycle threshold, the amount of the template in the reaction can be accurately determined. Such quantitative measurement is called Ct value quantification method.

Another PCR quantitative method is known as Digital PCR (Bert Vogelstein and Kenneth W. Kinzler; Digital PCR, Proc. Natl. Acad. Sci. USA, Vol. 96, pp. 9236-9241, 1999). In the digital PCR method, the test sample is distributed among numerous reaction wells, and the chance for the test sample in each reaction well to have more than one copy of template is less than 1. In other words, according to the possibility calculation, each reaction well can have no template or only a few copy of the template. Each reaction well goes through thermal cycles under PCR experimental conditions. Afterwards, each reaction well is assessed based on the detected signals (e.g. fluorescence signals) to determine whether the reaction well shows a positive signal (with a fluorescent signal higher than a selected threshold) or a negative signal (with a fluorescent signal lower than a selected threshold). The reaction well of the positive signal indicates that said reaction well has at least one copy of template and replication is successful in that reaction well. On the other hand, the reaction well of the negative signal indicates that said reaction well has no template or the replication of the existing template has failed in that reaction well. In the digital PCR method, the quantitative result is calculated using the total number (N) of the reaction wells in that experiment, the number (n⁻) of reaction well(s) of negative signal (i.e. negative wells) after the reaction, and the number (n⁺) of reaction well(s) of positive signal (i.e. positive wells) after the reaction, to determine the concentration of template(s) in one batch of the test sample (FengShen, Wenbin Du, Jason E. Kreutz, Alice Fok and Rustem F. Ismagilov; Digital PCR on a SlipChip; Lab Chip, 2010; DOI: 10.1039/c004521g).

The Ct value quantitative method described above is suitable for a test sample of template(s) having higher concentrations, while the digital PCR method is useful for a test sample of the template(s) having a wide concentration range. The concentration range of the template(s) measured by the traditional qPCR method may span 5 orders of magnitude. For the digital PCR method, the concentration range of the template(s) to be measured depends on the number of reaction wells. When the concentration range of the template(s) to be measured span over 6 orders of magnitude, then, 1,000,000 reaction wells need to be used. However, if only 10 to 1,000 reaction wells are involved as in the ordinary experiments, the concentration range of the template(s) to be measured may only span over 1˜3 orders of magnitude.

The following descriptions are provided to further define the present invention for illustration purposes.

A specimen or sample generally refers to the object being tested. For example, the sample may be an agricultural specimen, pathological slices, a soil specimen or the like, which contains nucleic acid fragments (DNAs or RNAs). A template refers to a strand of DNA or RNA or microRNA with a specific sequence, which is also called biomarker and can be detected via PCR amplification.

A test or a test item may refer to one or more test or test items performed to a specific template and the test reagent may refer to a formulation of several ingredients including primer pair(s) and optionally probes used for the specific template in a particular experiment or test. Generally, one test item is used to check one template.

A test panel may refer to a comprehensive examination of one specimen by performing more than one test item at the same time on the same specimen, which detects a variety of templates (template molecules). Comprehensive test results for the template molecules in the specimen may be used as a basis for a specific diagnosis of a disease, for example. For the inspection of a medical specimen, an agriculture specimen or an environment specimen, the examination of the concentrations of several template molecules may be required for the diagnosis or analysis. For example, in the medical examination of a pathological specimen, the expression amount of RNA and microRNA is commonly used as a diagnostic marker (biomarker). Also, the quantity of various gastrointestinal bacteria can be used as health status indicators.

PCR array slide may refer to a slide plate containing a plurality of reaction wells, and each reaction well is to perform a PCR test. Generally, because the reaction wells are small (less than 1 microliter/well, even less than 0.01 microliter/well), such slide is often referred to as a micro-well PCR array or a nano-well PCR array.

In this invention, the number of the reaction wells and the types or sizes of the reaction wells on the PCR array plate having numerous reaction wells are individually allocated to measure different types of templates of diverse concentration ranges. Such an arrangement of the PCR plate renders the qualitative and quantitative analysis of different types of templates of divergent concentration ranges in one experiment possible. Hence, by using the measuring method of this invention, the same test panel of various assays may be performed in only one test slide. According to the measuring method for nucleic acid samples, a test slide (an assay array plate) with numerous reaction wells, up to several hundreds of reaction wells is provided. The test sample may be a nucleic acid sample, including more than one nucleic acid templates (targets). The measuring method according to this invention is especially suitable for measuring the sample having more than one nucleic acid templates, where the concentration ranges of various types of templates are very different. Among the PCR array plate having numerous reaction wells, different sets of reaction wells are individually allocated to measure different types of templates of diverse concentration ranges. Basically, the expected concentration of the template in a given test sample determines how many reaction wells allocated to measure such template. The test sample(s) may be loaded into these reaction wells, and the same volume or different volumes of test sample(s) may be dispensed into these reaction wells. For each reaction well, one or multiple sets of PCR reactions may be performed independently and simultaneously under one PCR test condition, if one or multiple pairs of primers and multi-sets of fluorescence labels are loaded into each reaction well.

In the conventional test panel, if large concentration-range differences exist among the target template molecules in the specimen, the specimen often needs to be concentrated or diluted to accommodate different target templates. Taking gene expression profiling as an example, for high-expression RNA, thousands to tens of thousands of copies (1,000˜100,000 copies/cell) may be present in the cell; for moderate-expression RNA, tens of thousands of copies (10˜10,000 copies/cell) may be present in the cell; and for low-expression RNA, only a very small number of copies (0˜100 copies/cell) may be present in the cell. If the test panel aims at the target templates including high-, medium- and low-expression RNA or microRNA, the specimen must be treated with dilution or concentration differently for different target molecules, which significantly increases the complexity of the test panel.

The present application now will be described more fully hereinafter with reference to the following examples, which are representative of the certain exemplary embodiments but which are not to be construed as limiting the scope of the application. In one exemplary embodiment of the application, three targets T1, T2, T3 need to be measured in a test specimen. Based upon the knowledge on the origin of the specimen, the most likely concentration ranges of T1, T2 and T3 in the extracted solution (i.e. the solution containing nucleic acids extracted from the specimen) are known to be 10,000,000˜100,000 copies per micro-liter, 100,000˜100 copies per micro-liter, and 1,000˜0 copies per micro-liter, respectively. If a typical real time PCR reaction 96 titer plate, which using 10 micro-liter of extracted solution in each vial and having a quantitative range of 500˜1,000,000 copies/vial, is used to perform the test. If the extracted solution is applied directly, the possible copy number in the vial would be 100,000,000˜10,000,000 copies, 1,000,000˜1,000 copies, and 10,000˜0 copies, for T1, T2, and T3, respectively. Only T2 will be fully covered by the quantitative range of this test condition. To measure T1 accurately, the extracted solution needs to be diluted 100 times. To measure T3 accurately, the extracted solution needs to be concentrated at least 100 times. Therefore, three separate tests with one dilution operation and one concentration operation are required. In such case, the current approach is to set the templates T1, T2, T3 at different dilution rates, individually for traditional qPCR or digital PCR tests, thus causing inconvenience to the user with extra steps.

The present invention takes advantages of the particular arrangement of the reaction wells in the PCR array slide configured for concentration differences of various target templates in the specimens, so that the target templates can be detected simultaneously in one array slide for one test assay or test panel, without the need of pre-treating (concentrating or diluting) the specimen.

The present invention can easily adjust the number and the volume of the reaction wells in the PCR array slide assigned for each target template for PCR reaction(s), so that the target templates can be detected simultaneously in one array slide and the desired information for one test assay or test panel can be provided at one time. For the measuring method of the present invention, a PCR array slide having a number of (hundreds or more) reaction wells may be used. The test sample may be applied to the reaction well with the same or different volumes in each well. However, of each reaction well one or a group of PCR reactions may be performed independently. The number of the reaction wells assigned for each assay depends on the estimated copy numbers of the template molecules present in the sample.

Following the above mentioned case and using the method provided by this invention, a slide with 2,500 wells and each well may be loaded 10 nano-liter of extracted solution. The slide is suitable for measuring the target of 100,000˜0 copies per well accurately. If 10 nano-liter of the extracted solution is loaded into one of the well on the slide, the possible copy numbers of three targets T1, T2, T3 in one well would be 100,000˜1,000 copies, 1,000˜1 copies, and 10˜0 copies per well, respectively. The concentration ranges of three targets T1, T2 and T3 are within the quantifiable range, but the concentration ranges of the targets T1/T2 and T3 are very different and would be difficult to be quantified accurately under this condition. Therefore, based on the method of this invention, at least one well is assigned to measuring T1, one well is assigned to measuring T2, and the remaining wells (2,500-2) are assigned to T3. The primers and probes to detect T1, T2, and T3 are pre-dispensed into the wells according to above design. The slide is then loaded with the extracted solution and a real time PCR reaction is performed. The fluorescence intensity of each well at each cycle is measured and recorded. The data is then analysed based on the Ct value quantification method. The concentrations of T1 and T2 can be measured accordingly. The wells assigned to T3 is first analysed by the Ct value quantification method. Then, whether all the wells are able to be determined with a Ct value are checked. The wells with a valid Ct value imply a certain amount of targets are detected inside wells, and are defined as positive wells. The wells which can not generate a valid Ct value imply no targets are detected inside wells, and are defined as negative wells. Alternatively, wells with an end-point fluorescence signal higher than a selected threshold value can be defined as positive wells. Wells with an end-point fluorescence signal lower than a selected threshold value can be defined as negative wells. The threshold value depends on the instrument and can be determined by standard target samples. Three possibilities might happen to the wells assigned to T3: 1) all the wells are positive wells, 2) some of the wells are positive wells, and the other wells are negative wells; and 3) all of the wells are negative wells. In the first case, the Ct values from all the wells assigned to T3 are statistically averaged and a most likely concentration of T3 based on the average result is reported. In the second case, the number (n⁺) of the positive wells and the number (n⁻) of the negative wells are used to calculate the average chance (p) of finding at least one T3 targets in a well, that is n⁺/(n⁺+n⁻). The chance (p) is then used to estimate the most likely concentration of T3 presented in the extracted solution. In the third case, T3 has zero copy or close to zero copy. Accordingly, one slide and one PCR test is able to measure the targets with wide spread in concentration ranges without extra dilution or concentration steps.

Owing to the existence of deviations in laboratory operations, more than one well can be assigned to T1 and T2, says 10 wells for T1 and 50 wells for T2, to perform the redundant assay and to improve the precision of the measurement by using the statistical average of the redundant Ct value. However, assigning N wells to T3 actually extends the detection limit of the test to 1/N of that when a single well is used, theoretically.

An assay array slide plate having N (the number of) reaction wells of the same volume is provided. The templates to be determined or tested are respectively T1, T2 and T3. Under the normal conditions, the test sample is loaded into each reaction well, and each reaction well receives the same volume v of the test sample. One or more sets of PCR primer pairs and detection primers are loaded into individual reaction well according to predetermined PCR assays, allowing the individual reaction well to have the corresponding template(s) amplified.

For example, in the test sample fluid of the volume v, the copy number of the template T1 may range from 1000 to 100,000, while the copy number of the template T2 may range from 1 to 10,000 and the copy number of the template T3 may range from 0.01 to 100. In this invention, the number of reaction wells allocated for different templates are adjusted, for example, 10 reaction wells are allocated to receive the template T1 and filled with the primer probes of the template T1, 100 reaction wells are allocated to receive the template T2 and filled with the primer probes of the template T2, and 1,000 reaction wells are allocated to receive the template T3 and filled with the primer probes of the template T3. That is, N reaction wells equal to 1,110 reaction wells.

It is noted that the number of the reaction wells for control groups in PCR experiments as the quantity reference may be omitted herein for the conveniences of descriptions.

During the experiment, the test sample solution is dispensed and distributed among 1,110 reaction wells for individual PCR reactions. After the PCR reactions, the fluorescence signal of each reaction well is recorded. The recorded fluorescence signals are plotted as fluorescent curves and are used to calculate the concentration of the template(s) in the test sample according to the below steps.

Step 1: Determine whether the result (i.e. the fluorescent signal) is a positive or negative signal for each reaction well.

Step 2: If the result is a negative signal for a given reaction well, the given reaction well is recorded as of a negative signal. If the result is a positive signal for a given reaction well, the copy number of the template inside the given reaction well at the beginning of the reaction is estimated according to the quantitative qPCR methods.

Step 3: Among the total number of reaction wells of the template T1 (N_T1), the number of reaction wells showing negative results (n_T1) is obtained and used to estimate the expected copy number of the template T1 in each reaction well through statistical methods such as Poisson distribution.

Step 4: For the reaction wells showing positive results for T1 templates, qPCR quantitative methods, such as Ct value quantification method is used to estimate the copy number of the template T1 in each reaction well.

Step 5: The results from steps 3 and 4 are used to estimate the concentration of the template T1 (T1 template) in the test sample through the established methods.

Step 6: The steps 3˜5 are repeated to estimate the concentration of templates T2 and T3 (T2 and T3 templates).

If 1.6 copies of the template exist inside a reaction well, the possibility of a positive result (positive signal) is 0.8. When the copy number of the template is more than 5 inside the reaction well, the possibility of the positive result is almost 1.0.

In an experiment using N reaction wells filled with a specific primer pair, if all of N reaction wells show positive results, the Ct value quantification method or other similar evaluation methods is used to calculate the estimated copy number of the template in each reaction wells at the beginning of the experiment. Then, the average value of the estimated copy number of the template for N reaction wells is obtained by statistical methods.

### Experimental Example I

The PCR array slide having 10,000 reaction wells is provided, and each reaction well is loaded with the test sample of a volume v of 0.01 microliter (0.01 microliter/well). The test sample (pathological specimen) includes three templates (nucleic acid molecules) T1, T2, T3 (the target templates). In the test sample, the expected ranges of the copy numbers of these three target templates are as follows:

T1 template molecules are expected to be 10,000 to 1,000,000 molecules per microliter (10,000˜4,000,000 copies/μL)

T2 template molecules are expected to be 10 to 100,000 molecules per microliter (10˜100,000 copies/μL)

T3 template molecules are expected to be 0.1 to 1,000 molecules per microliter (0˜1,000 copies/μL)

Each reaction wells is analyzed based on Ct value quantification method and its quantitative range is from 10 to 100,000 molecules.

At first, the sample is loaded into the reaction well(s) (v=0.01 μL), and within the reaction well(s), the copy numbers of the template T1 ranges between 100 to 10,000 molecules (100˜10,000 copies/well), the copy numbers of the template T2 ranges between 0.1 and 1,000 molecules (0.1˜1,000 copies/well) and the copy numbers of the template T3 ranges between 0.001 to 10 molecules (0.001˜10 copies/well).

Based on the Ct value quantitative method, the template T1 can be directly measured quantitatively, the template T2 has to be concentrated at least 100-fold, the template T3 has to be concentrated at least 10,000 times. Hence, at least three different tests are to be carried out. For the digital quantitative method, the template T1 has to be diluted at least 10,000 times, the template T2 has to be diluted 1000-fold and the template T3 has to be diluted 10-fold.

Different numbers of react wells are allocated for various templates. Among the 10,000 reaction wells, 8,000 wells are allocated to the tests of the template T3, 1,880 wells are allocated to the tests of the template T2 and 120 reaction wells are allocated to the tests of the template T1.

If the copy number of the template T1 ranges between 100˜40,000 copies/well in each of the reaction wells, the Ct value quantification method can be used for quantification and the statistical average value of the quantitative results of 120 reaction wells are taken as the copy number of the template T1 obtained from the sample. If the copy number of the template T2 per reaction well ranges between 0.1˜1,000 copies/well, when the copy number of the template T2 is greater than 5 copy/well, the Ct value quantification method can be used for quantification and the statistical average value of the quantitative results of all the 1,880 reaction wells are taken as the copy number of the template T2 obtained from the sample. Alternatively, when the copy number of the template T2 is less than 5 copies/well, the digital PCR quantitative method is used to measure the number (quantity) of the reaction wells with positive signals among the 1,880 wells and the statistical method is used to calculate the possible copy number in each reaction well. The template T3 is measured similarly to the template T2. When the copy number of the template T3 is greater than 5 copies/well, the Ct value quantification method can be used for quantification and the statistical average value of the quantitative results of all the 8,000 reaction wells are taken as the copy number of the template T3 obtained from the sample, or when the copy number of the template T3 is s less than 5 copies/well, the digital PCR quantitative method is used to measure the number (quantity) of the reaction wells with positive signals among the 8,000 wells and the statistical method is used to calculate the possible copy number in each reaction well. Therefore, no dilution or concentration is required to pre-treat the templates and the three templates of three very different concentration ranges can be measured quantitatively at the same time by assigning different numbers of reaction wells for these three templates.

In the foregoing example, the Ct value quantitative method is not very accurate for measuring the template of the copy number ranging from 1 to 100. It is better to use the statistical results of two or more reaction wells. According to the statistical analysis, the average of the optical signal is firstly determined and then the Ct value is calculated; alternatively, the Ct values of the reaction wells are calculated and then the average thereof are determined. The statistical average value may be the arithmetic mean value, median value, or other statistical significant values.

In the preceding example, before making any quantitative calculation, a screening step may be performed to remove the abnormal reaction wells, i.e. the reaction wells ongoing abnormal reaction, the reaction wells not properly filled with the sample or the probe.

In the preceding example, the reaction wells of the array slide may be spared and may not be used as blanks or as a baseline reference for optical reaction or biochemical reactions or as negative controls.

For the allocation of the reaction wells, the lower the copy number of the target template is, the more reaction wells are required for the target template. In contrast, the higher the copy number of the template is, the less reaction wells (even one reaction well) are needed. However, for considering the errors of the measurements, a plurality of reaction wells (e.g. 10 wells) may be used to facilitate the average values to be taken.

If the copy number of the target template is higher than the upper limit of quantification based on the Ct value quantification method, the volume or the amount of the sample to be filled in the particular reaction well may be reduced. For example, a normal reaction well is loaded with 0.01 microliter (μL) of the sample and the particular reaction well is loaded with 0.002 microliter (μL) of the sample.

If the copy number of the target template is too low, large quantities of reaction wells are required for digital PCR quantification, and the volume or the amount of the sample to be filled in the particular reaction well may be increased. For example, a normal reaction well is loaded with 0.01 microliter (μL) of the sample and the particular reaction well is loaded with 0.05 microliter (μL) of the sample.

The PCR array slide as well as the number of the reaction wells of the slide may be specially designed according to the requirements of the tests or the test panel.

For just a few PCR reaction tests, the controls may be incorporated as references for quantification and the probe sets can be added to the slide.

In general, the minimum number of the reaction wells can be estimated using the reciprocal of the lower limit of the copy number of the target template. The estimation method is described as follows:

If the reaction well is loaded with 0.02 μL of the sample (0.02 μL sample/well) and the copy number of the target template Ti ranges between 0.001 to 100 copies (0.001˜100 copies/well). The lower limit of the target template Ti is 0.001 copy/well, its reciprocal is 1/0.001=1,000. That is, the minimum number of the reaction wells is 1,000 (i.e. 1,000 reaction wells). Taking the errors into considerations, having 1,200 or more reaction wells is preferred.

The data analysis step after the PCR assay(s) may be performed as follows:

At the end of the reaction, the concentration of the target template in the sample is calculated according to the fluorescence curve of each of the reaction wells following the below procedures:

The total number of the reaction wells allocated to the target template Ti is assumed to be N. Among the N reaction wells, the number n⁺ of the positive reaction wells (with positive signals) and the number n⁻ of the negative reaction wells are measured and determined. If the n⁺/N>a pre-defined number, Criteria U, the Ct value quantitative method is used to determine the initial copy number of the reaction wells. If the n⁺/N<a pre-defined number, Criteria L, the digital PCR quantitative method is used to determine the initial copy number of the reaction wells. Criteria U can be 99%, 95%, or 90%, and Criteria L can be 95%, 90%, or 85% accordingly.

Also, for the data analysis step, a checking step may be incorporated to check whether the reaction well is an effective reaction well and to remove the invalid reaction well in advance. Such step may be performed as follows:

Among the N reaction wells, the number n0 of the invalid reaction well(s) the number n⁺ of the positive reaction wells (with positive signals) and the number n⁻ of the negative reaction wells are measured and determined. If n⁺/(N−n0)>Criteria U, the Ct value quantitative method is used to determine the initial copy number of the reaction wells. If n⁺/(N−n0)<Criteria L, the digital PCR quantitative method is used to determine the initial copy number of the reaction wells.

Alternatively, such step may be performed as follows:

Among the N reaction wells, the number n0 of the invalid reaction well(s) the number n+ of the positive reaction wells (with positive signals) and the number n⁻ of the negative reaction wells are measured and determined. If n⁺/(N−n0)>Criteria U, the Ct value quantitative method is used to determine the initial copy number of the reaction wells. If n⁺/(N−n0)<Criteria L, the digital PCR quantitative method is used to determine the initial copy number of the reaction wells. If n⁺/(N−n0)<Criteria U and n⁺/(N−n0)>Criteria L respectively, Ct value quantification method and digital PCR quantitative method are individually used and the results are weighted to get the most possible copy number.

Through adjusting the number of reaction wells in various sets of reaction wells for different types of templates, it is possible to use one slide in one experiment to determine the concentrations of different types of templates, of which the concentration variation is up to 3˜7 orders of magnitude, without the need of multiple dilution processes and multiple experiments on the given test sample.

The reaction wells within the test slide or assay array plate may be designed to be of the same volume, the same size or of different volumes or sizes. For example one set of reaction wells may be designed to have a volume of 2.8 nL while the other set of reaction wells may be designed to have a volume of 16 nL.

Each reaction well may be filled with a certain volume of the primer probe solution, and such volume can be adjusted based on experimental protocols. Using the reaction well having 2.8 nL volume as stated above, the volume of the primer probe solution loaded into the reaction well may be controlled to be within the range of 0.02 nL to 2.5 nL. Using the reaction well of 16 nL as an example, the volume of the primer probe solution may be within the range of 0.02 nL to 15 nL. For a reaction well of 2.8 nL pre-filled with 1 nL of the primer probe solution, 1.8 nL fluid volume of the test sample is received in the reaction well. For the reaction well of 16 nL and pre-filled with 1 nL of the primer probe solution, this reaction well may have 15 nL of the test sample.

By choosing in advance the volume of the reaction well, the number of reaction wells, the volume of the pre-filled primer probe solution, it is possible to well control the dilution ratio or the copy numbers of one or more template(s) within the reaction well, so as to make sure the concentration ranges of one or more templates at least partially overlap.

For example, one approach is to fill different volumes of the primer solution into the same set of reaction wells for the single template T4. Also, in the set of reaction wells for the single template T4, 10 reaction wells of 18 nL in volume and 1000 reaction wells of 2.8 nL in volume are included. In the 10 reaction wells of 18 nL, three wells are pre-filled with 1 nL of the primer probe solution while 7 wells are pre-filled with 15 nL of the primer probe solution. All of the 1000 wells of 2.8 nL are pre-filled with 0.1 nL of the primer probe solution.

Not only the primer probe solution to be prefilled into the reaction wells for a specific template may be adjusted, but also the concentration of the primer probe solution may be adjusted to make sure that the possible concentration ranges of the specific templates within the sample are overlapped.

For quantitative PCR (qPCR) based on Ct value quantification method, at least one copy of the template is present in the sample fluid filled into the reaction well for PCR. Hence, the lower threshold of this experiment is one copy of the (target) template, denoted as a concentration of 1/Vs (Vs: sample volume). For the reaction well having the sample fluid of 2 nL, the lower threshold of concentration is 1 target template/2 nL. If the concentration of the target template is lower than the lower threshold, no results will be obtained using the traditional PCR method, or concentration of the sample fluid is required to increase the concentration of the target template. However, in this invention, a great number of reaction wells (10, 100, 1000 or more reaction wells) may be allocated for one or more templates for PCRs. The numbers of the reaction wells showing positive results and negative results are used to calculate the concentration(s) of the targeted template(s).

A more generalized description of this invention is that a sample having m types of nucleic acid targets (m targets) to be measured and a slide with n number of reaction wells are provided. Each well can accurately quantify D-lower copies to D-upper copies of target in a PCR reaction, wherein m is an integer greater than or equal to two, while n can be 2,500, 10,000, or 40,000, D-lower is a integer greater than or equal to one, and D-upper usually ranges from 10⁶ to 10⁹. The optimized scheme of allocating reaction wells to targets is based on the concentration ranges to be covered by this slide. For target i among the m targets, wherein i is an integer ranging from 1 to m, Cᵢ-upper and Cᵢ-lower are the upper and lower detection limits of the target i intended to be covered by this slide. The theoretical probability of finding at least one copy in a well is pᵢ=(Cᵢ-lower×v), wherein v represents the sample volume loaded to a well. To detect at least one positive well in the test, theoretically at least 1/pᵢ wells on the slide for measuring the target i are needed.

For the target i, to prevent the target present in all wells from exceeding D-upper, at least one well should be loaded with the sample volume smaller than D-upper/Cᵢ-upper.

For the target(s) only needs one well, several wells can be assigned to the target(s) as technical redundant to eliminate a large deviation caused by experimental error. The typical number for technical redundant is 2 to 10 wells. For those targets already using more than 10 wells, technical redundant may not be necessary.

The fluorescence intensity data recorded during the PCR cycle is analysed according to the following steps:
1). Check and determine whether the well is a good well; if so, mark the well as a good well; if not, mark the well as a bad well and be eliminated from the following analysis. A good well means a well where PCR data can be obtained. A bad well means a well where PCR data cannot be obtained, mostly due to instrumental or operational errors. Results of good wells can be later interpreted as “negative” or “positive”, based on 2 different methods (Ct or threshold).
2). Check if a Ct value can be determined for each good well; if so, mark the well as a positive well and estimate the Ct value for that well; if not, mark the well as a negative well. Failure to generate a valid Ct value for a certain well can be due to insufficient fluorescence amplification after PCR. Alternatively, compare end-point fluorescent intensity of each good well to a threshold value. The positive wells can be wells with end-point fluorescent intensity higher than a threshold value, and the negative wells can be wells with end-point fluorescent intensity lower than a threshold value.
3). Count the number (n⁺, n⁻) of positive and negative wells for each target.
4). Calculate the target concentration as follow:
a). if (n⁻/(n⁺+n⁻)) is less than a pre-selected ratio (R+), estimate the target concentration in the tested sample via the statistical averaging of the Ct values from all positive wells, wherein R+ can be 1%, 5%, or 10%;
b). if (n⁻/(n⁺+n⁻)) is greater than a pre-select ratio (R−), estimate the target concentration in the tested sample by calculating (n⁺/(n⁺+n⁻)) using a statistical probability distribution method, wherein R− can be 5%, 10%, or 15%;
c). if (n⁻/(n⁺+n⁻)) is between the above two pre-selected ratios, the target concentration can be estimated by taking a weighting average of the concentrations determined from steps a) and b).
d). if (n⁺)=0, report that the target concentration is lower than detection limit of this slide.

The pre-select ratios R+ and R− can be determined by performing calibration process using a set of standards. It is also possible setting a fluorescent threshold to determine whether a good well shall be considered as a positive well or a negative well. The threshold can be an absolute intensity or the percentage change of the intensity. The threshold can be determined by a calibration process using a set of standards. The statistical distribution may be Poisson or binominal distribution.

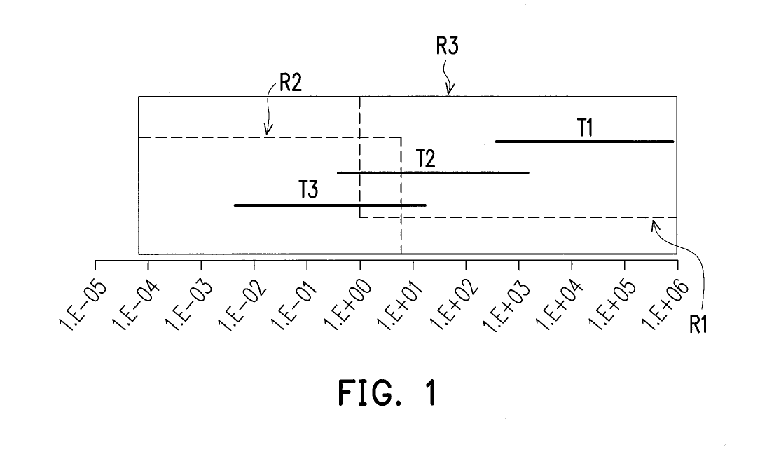

According to the measuring method of the present invention, the concentration ranges of the target template in a given sample that may be quantified are expanded. As shown in FIG. 1, the concentration ranges of the three different templates T1, T2 and T3 are overlapped with the quantitative concentration ranges of conventional qPCR (marked as R1) and digital qPCR (marked as R2). The quantitative concentration range (marked as R3) of the measuring method of the present invention may be extended beyond the quantitative concentration ranges R1, R2 of conventional qPCR and digital qPCR.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

What is claimed is:

1. A method of measuring concentrations of more than one template in a sample, comprising:
providing a test slide having a plurality of reaction wells;
providing the sample having m targets to be measured, wherein each of the plurality of reaction wells is loaded with a volume v of the sample;
providing an upper concentration range Cᵢ-upper and a lower concentration range Cᵢ-lower of each of the m targets intended to be measured with the test slide;
calculating a probability (pᵢ) of finding at least one copy of the target i in one reaction well of the plurality of reaction wells;
determining a number of the reaction wells of the plurality of reaction wells to be allocated to the target i of the m targets, wherein the number of the reaction wells of the plurality of reaction wells to be allocated to the target i is at least 1/pᵢ; and
dispensing primer-pairs corresponding to the m targets to the plurality of reaction wells,
wherein m is an integer greater than or equal to two.

2. The method of claim 1, further comprising:
performing a PCR (polymerase chain reaction) test and recording a fluorescence intensity from each reaction well of the plurality of reaction wells for each PCR cycle; and
estimating a concentration of the target i of the m targets by:
counting a number (n+) of positive wells in the number of the reaction wells of the plurality of reaction wells to be allocated to the target i of the m targets;
counting a number (n−) of negative wells in the number of the reaction wells of the plurality of reaction wells to be allocated to the target i of the m targets;
if none of the reaction wells of the plurality of reaction wells to be allocated to the target i of the m targets is determined to be the negative wells, estimating the concentration of the target i of the m targets presented in the sample via cycle threshold values of the positive wells;
if 5% to 15%, or above, of the reaction wells of the plurality of reaction wells to be allocated to the target i of the m targets are determined to be the negative wells, estimating the concentration of the target i of the m targets presented in the sample via a probability distribution of n⁺/(n⁺+n⁻); and
if none of the reaction wells of the plurality of reaction wells to be allocated to the target i of the m targets is determined to be the positive wells, reporting the concentration the target i of the m targets presented in the sample as lower than the lower concentration range Cᵢ-lower,
wherein n⁺ and n⁻ are respectively an integer greater than or equal to zero.

3. A method of measuring concentrations of more than one template in a sample, comprising:
providing a test slide having n reaction wells;
providing a sample having m targets to be measured, wherein each of the n reaction wells is loaded with a same volume v of the sample;
providing an upper concentration range Cᵢ-upper and a lower concentration range Cᵢ-lower of a target i of the m targets intended to be measured using the test slide;
determining a number of the n reaction wells to be allocated to the target i of the m targets by calculating a probability (pᵢ) of finding at least one copy of the target i in one of the n reaction wells, wherein the number of the n reaction wells to be allocated to the target i is proportional to 1/pᵢ; and
dispensing primer-pairs corresponding to the m targets to the n reaction wells,
wherein m is an integer greater than or equal to two and n is a positive integer.

4. The method of claim 3, further comprising:
performing a PCR (polymerase chain reaction) test and recording a fluorescence intensity from each reaction well of the plurality of reaction wells for each PCR cycle;
estimating a concentration of the target i of the m targets by:
counting a number (n+) of positive wells in the number of the reaction wells of the plurality of reaction wells to be allocated to the target i of the m targets;
counting a number (n−) of negative wells in the number of the reaction wells of the plurality of reaction wells to be allocated to the target i of the m targets;
if none of the reaction wells of the plurality of reaction wells to be allocated to the target i of the m targets is determined to be the negative wells, estimating the concentration of the target i of the m targets presented in the sample via cycle threshold values of the positive wells;
if 5% to 15%, or above, of the reaction wells of the plurality of reaction wells to be allocated to the target i of the m targets are determined to be the negative wells, estimating the concentration of the target i of the m targets presented in the sample via a probability distribution of n⁺/(n⁺+n⁻); and
if none of the reaction wells of the plurality of reaction wells to be allocated to the target i of the m targets is determined to be the positive wells, reporting the concentration the target i of the m targets presented in the sample as lower than the lower concentration range Cᵢ-lower,
wherein n⁺ and n⁻ are respectively an integer greater than or equal to zero.

5. A method of measuring concentrations of more than one template in a sample, comprising:
providing a test slide having a plurality of reaction wells;
providing a sample having m targets to be measured;
providing an upper concentration range Cᵢ-upper and a lower concentration range Cᵢ-lower of the target i of the m targets intended to be measured using the test slide;
providing an upper limit N-upper of copies of the target i of the m targets to be quantified via one reaction well of the plurality of reaction wells, wherein a maximum volume to be loaded to the one reaction well for measuring the target i of the m targets is N-upper/Cᵢ-upper; and
dispensing primers to the test slide according to the m targets.

6. The method of claim 5, further comprising:
performing a PCR (polymerase chain reaction) test and recording a fluorescence intensity from each reaction well of the plurality of reaction wells for each PCR cycle;
estimating a concentration of the target i of the m targets by:
counting a number of positive wells (n⁺) in the number of the reaction wells of the plurality of reaction wells to be allocated to the target i of the m targets;
counting a number of negative wells (n⁻) in the number of the reaction wells of the plurality of reaction wells to be allocated to the target i of the m targets;
if none of the reaction wells of the plurality of reaction wells to be allocated to the target i of the m targets is determined to be the negative wells, estimating the concentration of the target i of the m targets presented in the sample via cycle threshold values of the positive wells;
if 5% to 15%, or above, of the reaction wells of the plurality of reaction wells to be allocated to the target i of the m targets are determined to be the negative wells, estimating the concentration of the target i of the m targets presented in the sample via a probability distribution of n⁺/(n⁺+n⁻); and
if none of the reaction wells of the plurality of reaction wells to be allocated to the target i of the m targets is determined to be the positive wells, reporting the concentration the target i of the m targets presented in the sample as lower than the lower concentration range Cᵢ-lower,
wherein n⁺ and n⁻ are respectively an integer greater than or equal to zero.

7. A method of determining a nucleic acid target concentration via a plurality of PCR tests, comprising:
performing a same real time PCR reaction in a group of wells, wherein a number of the wells is greater than 10;
recording a fluorescence intensity of each well of the group of wells at each cycle;
analysing the fluorescence intensity of the each well, wherein the step of analysing the fluorescence intensity comprises:
checking the each well of the group of wells to be a good well or a bad well;
marking a well of the group of wells as the good well if the well of the group of wells is a good well, and marking the well as the bad well if the well of the group of wells is a bad well and eliminating the bad well;
checking for a cycle threshold (Ct) value for the good well of the group of wells; and
marking the good well as the positive well if the Ct value of the good well is determined and estimating the Ct value for the good well and marking the well as the negative well if the Ct value of the good well is unable to be determined;
determining a number (n⁺) of the positive wells and a number (n⁻) of the negative wells for each nucleic acid target; and
calculating the nucleic acid target concentration, wherein the step of calculating the nucleic acid target concentration comprises:
a) if the (n⁻/(n⁺+n⁻)) is less than a first pre-select ratio (R+), estimating the nucleic acid target concentration via a statistical averaging of the Ct values from all the positive wells;
b) if the (n⁻/(n⁺+n⁻)) is greater than a second pre-select ratio (R−), estimating the nucleic acid target concentration by applying a Poisson distribution of (n⁺/(n⁺+n⁻));
c) if n⁻ is between the above the first and the second pre-select ratios, the nucleic acid target concentration is estimated by taking a weighting average of the nucleic acid target concentration determined from the step a) and the nucleic acid target concentration determined from the step b); and
d) if n⁺=0, reporting the nucleic acid target concentration being lower than a detection limit, which has a concentration of 1/[(n⁺+n⁻)*v], wherein v is the sample volume for each well of the group of wells.
